(19) United States
(12) Patent Application Publication
(10) Pub. No.: US 2016/0217594 A1
(43) Pub. Date: Jul. 28, 2016

(54) COST-FUNCTION BASED METHOD AND APPARATUS FOR PROJECTION-DOMAIN BASIS DECOMPOSITION IN SPECTRAL COMPUTED TOMOGRAPHY

(71) Applicants: KABUSHIKI KAISHA TOSHIBA, Tokyo (JP); TOSHIBA MEDICAL SYSTEMS CORPORATION, Tochigi (JP)

(72) Inventors: Adam PETSCHKE, Lake Bluff, IL (US); Yu ZOU, Naperville, IL (US); Zhou YU, Palatine, IL (US); Xiaolan WANG, Buffalo Grove, IL (US)

(73) Assignees: KABUSHIKI KAISHA TOSHIBA, Tokyo (JP); TOSHIBA MEDICAL SYSTEMS CORPORATION, Tochigi (JP)

(21) Appl. No.: 14/603,135

(22) Filed: Jan. 22, 2015

(51) Int. Cl. G06T 11/00; A61B 6/00; A61B 6/03

(52) U.S. Cl. CPC G06T 11/003; A61B 6/032; A61B 6/482

(57) ABSTRACT

A global optimization and apparatus to decompose spectral computed tomography (CT) projection data into basis materials. A cost function is defined to represent the difference between the measured projection data and calculated attenuation data using projection lengths for the basis materials with corresponding material models and a detector model to calculate the detector response. The cost function may have many local minima and only one global minima. A global optimization method is then used to obtain the projection lengths corresponding to the global minimum of the cost function. The global optimization method can be either a single-stage optimization method, or can be performed in multiple stages, e.g., a first coarse optimization stage followed by a second fine optimization stage using the final values of the first stage as the inputs into the second stage. The global optimization method can be a stochastic optimization method.

### BACKGROUND

1. Field

This disclosure relates to decomposing spectral computed tomography (CT) projection data into basis-material components, and more particularly using a cost function and global optimization to solve for the basis-material components.

2. Description of the Related Art

Computed tomography (CT) systems and methods are widely used, particularly for medical imaging and diagnosis. CT systems generally create images of one or more sectional slices through a subject's body. A radiation source, such as an X-ray tube, irradiates the body from one side. A collimator, generally adjacent to the X-ray source, limits the angular extent of the X-ray beam, so that radiation impinging on the body is substantially confined to a planar region defining a cross-sectional slice of the body. At least one detector (and generally many more than one detector) on the opposite side of the body receives radiation transmitted through the body substantially in the plane of the slice. The attenuation of the radiation that has passed through the body is measured by processing electrical signals received from the detector.

Conventionally energy-integrating detectors have been used to measure CT projection data. Now, recent technology developments are making photon-counting detectors a feasible alternative to conventional energy-integrating detectors. Photon-counting detectors have many advantages including their capacity for performing spectral CT. To obtain the spectral nature of the transmitted X-ray data, the photon-counting detectors split the X-ray beam into its component energies or spectrum bins and count a number of photons in each of the bins. Since spectral CT involves the detection of transmitted X-rays at two or more energy levels, spectral CT generally includes dual-energy CT by definition.

Photon-counting detectors use semiconductors with fast response times compared to indirect detectors, such as scintillating crystals coupled to optical detectors (e.g., photo-multiplier tubes or avalanche photodiodes) to detect resultant scintillation photons. This fast response time enables photon-counting detectors to resolve in time individual X-ray detection events. However, at high X-ray flux rates indicative of clinical X-ray imaging, multiple X-ray detection events on a single detector can occur within the detector's time response—a phenomenon called pileup.

Semiconductor-based photon-counting detectors used in spectral CT can detect incident photons and measure photon energy for every event. However, due to the interaction depth and ballistic deficit, the measured photon energy cannot be related to incident photon energy uniquely. At high flux, pulse pileup may also result in lost counts.

Left uncorrected, pileup, detector nonlinearities, and other artefacts of the projective imaging process can degrade reconstructed images from photon-counting detectors. On the other hand, when these effects are corrected for or calibrated out of the data, spectral CT has many advantages over conventional CT. Many clinical applications can benefit from spectral CT technology, including improved material differentiation and beam hardening corrections. Moreover, compared with non-spectral CT, spectral CT extracts complete tissue characterization information from an imaged object.

Semiconductor-based photon counting detectors (PCDs) are promising candidates for spectral CT, capable of providing better spectral information compared with conventional spectral CT technology (e.g., dual-source, kVp-switching, etc.)

One challenge to more effectively using semiconductor-based photon counting detectors for spectral CT is performing the material decomposition from the projection data in a robust and efficient manner.

### BRIEF DESCRIPTION OF THE DRAWINGS

A more complete understanding of this disclosure is provided by reference to the following detailed description when considered in connection with the accompanying drawings, wherein:

FIG. 1 shows a flow diagram of an implementation of a material decomposition method using a cost function φ;

FIG. 2 shows a flow diagram of an implementation of a cost function minimizing process;

FIG. 3 shows a surface plot of a an implementation of a cost function for decomposing spectral CT data into material projection lengths corresponding to water and bone;

FIG. 4 shows a flow diagram of an implementation of a two-step material decomposition method; and

FIG. 5 shows a schematic diagram of an implementation of an X-ray CT apparatus having photon-counting detectors arranged in a fourth-generation geometry and energy integrating detectors (PCDs) arranged in a third-generation geometry; the CT apparatus further including control, processing, and data-acquisition circuitry;

FIG. 6 shows a schematic diagram of an implementation of an arrangement of PCDs in a predetermined fourth-generation geometry in a CT scanner apparatus;

FIG. 7 shows a schematic diagram of an implementation of an arrangement of PCDs in a predetermined fourth-generation geometry in combination with a detector unit in a predetermined third-generation geometry in a CT scanner apparatus; and

FIG. 8 shows a schematic diagram of an implementation of an arrangement of PCDs in a predetermined fourth-generation geometry in combination with two X-ray sources and two detector units in a predetermined third-generation geometry in a CT scanner apparatus.

### DETAILED DESCRIPTION

In one embodiment, there is provided an apparatus, comprising processing circuitry configured to (1) obtain projection data having a plurality of energy components, wherein the projection data represents an intensity of radiation having been transmitted through an imaged object and then detected at a plurality of detector elements; (2) calculate a cost function representing differences between the projection data and calculated data over the plurality of energy components, wherein the calculated data represents intensity of radiation transmitted through the imaged object, the calculated data being calculated using a detector model that approximates attenuation of the radiation, the detector model using a plurality of projection lengths, with each projection length corresponding to a respective material model of a plurality of material models; and (3) optimize the plurality of projection lengths until the calculated cost function converges to approximate a global minimum of the cost function.

In another embodiment, there is provided an apparatus, comprising: (1) an X-ray source radiating X-rays; (2) a plurality of detector elements each configured to detect a plurality of energy components of the X-rays that are radiated from the X-ray source and generate projection data; and (3) processing circuitry configured to (a) obtain the projection data having the plurality of energy components, wherein the projection data represents an intensity of radiation having been transmitted through an imaged object and then detected at the plurality of detector elements, (b) calculate a cost function representing differences between the projection data and calculated data over the plurality of energy components, wherein the calculated data represents intensity of radiation transmitted through the imaged object, the calculated data being calculated using a detector model that approximates attenuation of the radiation, the detector model using a plurality of projection lengths, with each projection length corresponding to a respective material model of a plurality of material models, and (c) optimize the plurality of projection lengths until the calculated cost function converges to approximate a global minimum of the cost function.

In another embodiment, there is provided a method, comprising: (1) obtaining projection data having a plurality of energy components, wherein the projection data represents an intensity of radiation having been transmitted through an imaged object and then detected at a plurality of detector elements; (2) calculating a cost function representing differences between the projection data and calculated data over the plurality of energy components, wherein the calculated data represents intensity of radiation transmitted through the imaged object, the calculated data being calculated using a detector model that approximates attenuation of the radiation, the detector model using a plurality of projection lengths, with each projection length corresponding to a respective material model of a plurality of material models; and (3) modifying the plurality of projection lengths until the calculated cost function converges to approximate a global minimum of the cost function.

In spectral CT, radiation having multiple energy components is used to make projective measurements of an object OBJ. These projective measurements are made at a series of angles enabling conventional CT image reconstruction methods similar to non-spectral CT. However, unlike non-spectral CT, spectral CT generates additional information that allows a decomposition of the projective measurements into several material components, usually two in current clinical settings. The material decomposition results in two component materials because there are two major differentiable interaction mechanisms resulting in X-ray attenuation as the X-ray beam traverses the imaged object OBJ. These interaction mechanisms are Compton scattering and photoelectric absorption. Mapping the projection data from the spectral domain to the material domain can be performed either before or after the image reconstruction process. However, performing material decomposition from the spectral domain to the material domain before the reconstruction process is preferable due to beam hardening considerations. Herein, we are concerned with performing the material decomposition before the image reconstruction process.

When most of the X-rays have energies well above the K-edge of the majority atoms of the imaged object OBJ, as is the case for conventional X-ray sources imaging biological objects, the material decomposition problem can be solved using only two energy components consistent with the existence of the two dominant interaction processes discussed above. Thus, spectral CT is sometimes referred to as dual-energy CT, and the material decomposition process can be referred to as dual-energy analysis. Herein, spectral CT will include at least dual-energy CT, but also includes projective measurements with more than two energy components, such that the two-material decomposition problem is overdetermined. As discussed in U.S. patent application Ser. No. 13/906,110, incorporated herein by reference in its entirety, the additional information provided by more energy components can be used effectively in noise balancing and related methods to improve image quality.

A dual-energy analysis method can be used because the attenuation of X-rays in biological materials is dominated by two physical processes (i.e., photoelectric absorption and Compton scattering). Thus, the attenuation coefficient as a function of energy can be approximated by the decomposition

μ(E,x,y)=μ_{PE}(E,x,y)+μ_{C}(E,x,y)

wherein μ_{PE}(E,x,y) is the photoelectric attenuation and μ_{C}(E,x,y) is the Compton attenuation. Alternatively, this attenuation coefficient can be rearranged into a decomposition of a high-Z material (i.e., material 1) and a low-Z material (i.e., material 2) to become

μ(E,x,y)≈μ₁(E)c₁(x,y)+μ₂(E)c₂(x,y),

where c₁(x,y) and c₂(x,y) are, respectively, the first and second basis images.

Next, a detector model of semiconductor-based photon counting detectors is discussed.

As discussed in U.S. patent application Ser. No. 13/866,965, incorporated herein by reference in its entirety, the response function of the radiation detectors can be calibrated to provide improved results. In one implementation, the detector model for the number of counts of each given radiation detector is

Nₘ=Tne^{-nτ}∫∫dEdE₀R₀(E,E₀)S(E₀)+Tn²e^{-nτ}∫∫∫dEdE₀dE₁R₁(E,E₀,E₁)S(E₀)S(E₁),

wherein each of the integrating time T, linear response function R₀, nonlinear response function R₁, and dead time τ are known for each radiation detector and energy component as a result of calibrations performed before the projective measurements on object OBJ. In the above nonlinear detector model only the first order nonlinear term is included. Generally, higher order nonlinear terms can also be included in the detector model for the number of counts. Each integral is integrated over the spectral range for the m^{th} energy bin. Thus, there is a unique count Nₘ for each energy bin/component of each detector.

The detected spectrum is given by

S(Eᵢ)=Sₐᵢᵣ(Eᵢ)exp[−μ₁(Eᵢ)L₁−μ₂(Eᵢ)L₂],

where the attenuation coefficients μ₁ and μ₂ are known functions of the X-ray energy, and the spectrum in the absence of an object OBJ (designated by Sₐᵢᵣ) is also known.

Similarly, the X-ray flux n for each detector is given by

n=nₐᵢᵣ∫dE₀S(E₀)exp[−μ₁(E₀)L₁−μ₂(E₀)L₂],

where nₐᵢᵣ is known. In one implementation, which is discussed more completely in U.S. patent application Ser. No. 14/103,137, incorporated herein by reference in its entirety, the value of nₐᵢᵣ is given by

nₐᵢᵣ=A·I_{ref},

where A is a calibration term unique to each detector that is determined before the projective measurements on object OBJ, and I_{ref} is the reference detector signal.

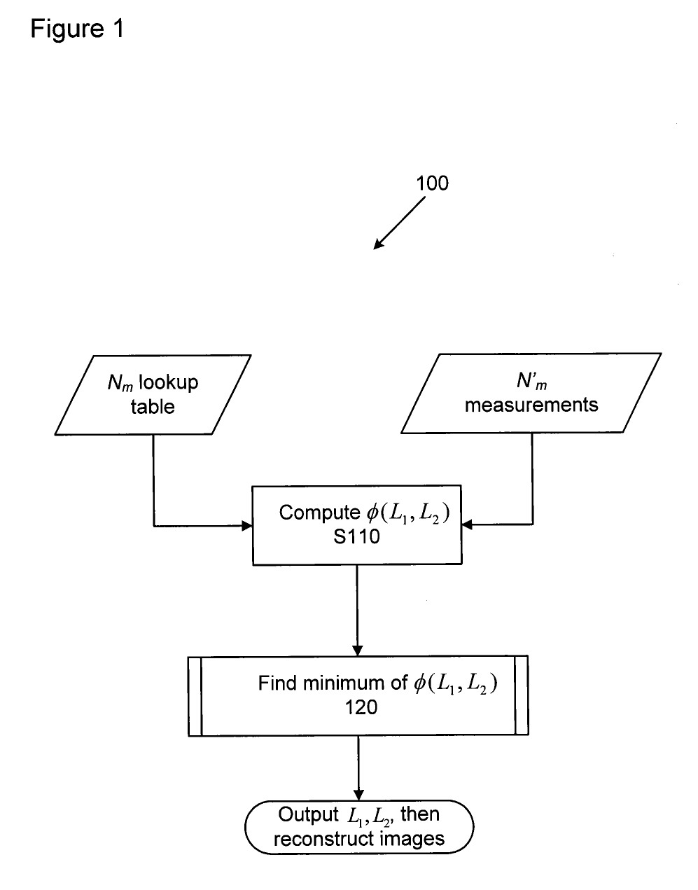

Referring now to the drawings, wherein like reference numerals designate identical or corresponding parts throughout the several views, FIG. 1 shows a method 100 to obtain the material decomposition projection lengths L₁ and L₂ using projection measurements combined with the detector model previously discussed.

The first step S110 of method 100 is to calculate a cost function φ(L₁,L₂). This cost function combines the measured projection data N′ₘ with corresponding calculated values Nₘ obtained from the detector model previously discussed. As shown in FIG. 1 the calculated values Nₘ using the detector model can be pre-computed and stored in a lookup table, or in an alternative implementation the values of Nₘ can be calculated at the time they are needed.

Several different cost functions (L₁,L₂) are possible. In one implementation, the cost function is the least squares of the difference between the measured counts N′ₘ and the calculated counts Nₘ, i.e.,

φ(L₁,L₂)=Σ(N′ₘ−Nₘ)².

In one implementation, the cost function is the weighted least squares of the difference between the measured counts N′ₘ and calculated counts Nₘ, i.e.,

$$ϕ ( {L}_{1 ,} {L}_{2} ) = \sum \frac{{( {N}_{m}^{′} - {N}_{m} )}^{2}}{{σ}_{m}^{2}} ,$$

where σₘ is the standard deviation of N′ₘ.

In one implementation, the cost function is the Poisson likelihood function, i.e.,

φ(L₁,L₂)=Σ[N′ₘ log(Nₘ)−Nₘ].

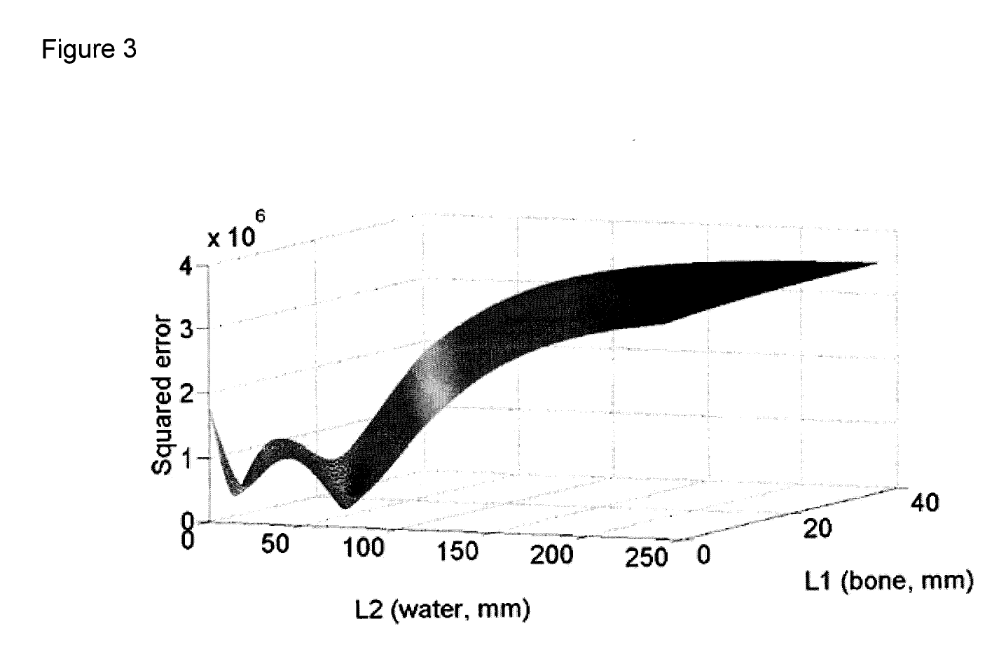

After computing the cost function in step S110, the method 100 proceeds to process 120 in which an optimization method is performed to find the minimum of the cost function. When the cost function has local minima that are different from the global minimum, a robust stochastic optimization process is beneficial to find the global minimum of the cost function. FIG. 3 shows an example of a least-squares cost function having multiple local minima and one global minimum. There are many known methods for finding global minima including: genetic algorithms, simulated annealing, exhaustive searches, interval methods, and other conventional deterministic, stochastic, heuristic, and metatheuristic methods.

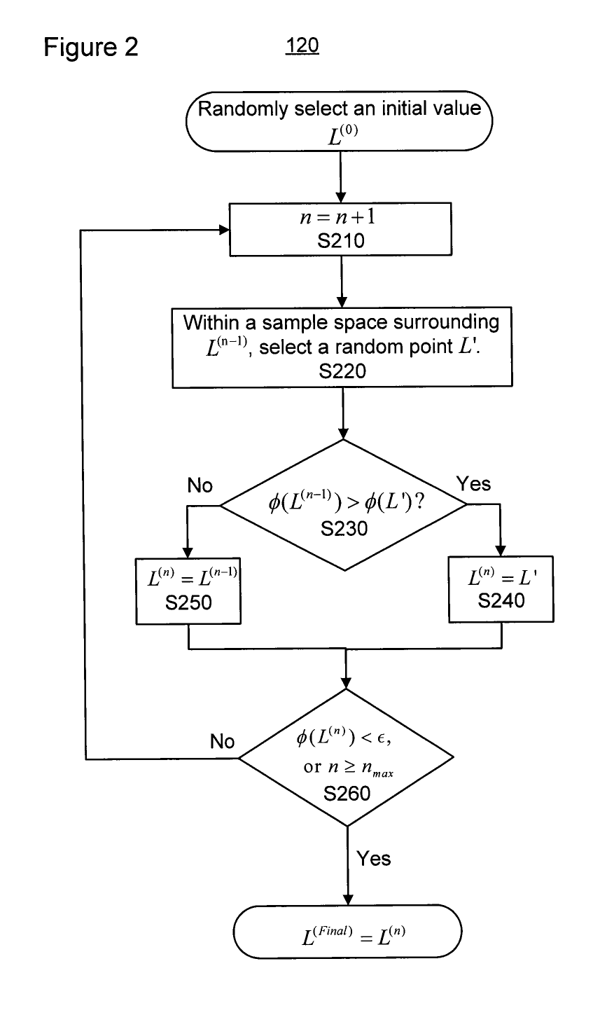

In one implementation, the method shown in FIG. 2 is used to perform the process 120. In FIG. 2, the process 120 starts when a random value is selected as the initial guess for L⁽⁰⁾=(L₁⁽⁰⁾,L₂⁽⁰⁾). Next, at step S210 the loop variable n is incremented.

Following step S210, the process 120 proceeds to step S220, wherein a new sample point L′ is randomly selected from the sample space surrounding the current set of projection lengths L₍ₙ₋₁₎=(L₁⁽ⁿ⁻¹⁾,L₂⁽ⁿ⁻¹⁾).

Proceeding to step S230, the process 120 inquiries as to which of value of the cost function φ(L⁽ⁿ⁻¹⁾)) or φ(L′) is smaller. In steps S240 and S250 the argument corresponding to the smaller value of the cost function is assigned as the next set of projection lengths L⁽ⁿ⁾=(L₁⁽ⁿ⁾),L₂⁽ⁿ⁾) for the next loop iteration.

Step S260 of process 120 evaluates whether the loop stopping criteria is satisfied. Although different stopping criteria can used, FIG. 2 shows an implementation wherein the loop stops when either a maximum number of loop iterations nₘₐₓ has been reached or the cost function falls below a predetermined threshold E. If the stopping criteria are satisfied, the process 120 exits the loop at S260 and reports the current projection length en)=(L₁⁽ⁿ⁾, L₂⁽ⁿ⁾) as the final projection length. Otherwise, the loop continues by proceeding from step S260 back to step S210.

In one implementation, the method 100 will be used with coarse searching criteria. For example, in the case that a grid of cost function values are pre-computed and assembled into a lookup table, then spacing of the grid for pre-computing cost function values will have a large spacing between the adjacent projection lengths that determine the grid of pre-computed cost function values. Alternatively, if the process shown in FIG. 2 is used without a pre-computed lookup table of cost function values, then a coarse search would be performed by using a large sample space surrounding the current projection length values from which to randomly select the new sample point L′. Additionally, the coarse search version of the implementation of process 120 shown in FIG. 2 will include that the stopping criterion threshold E will be larger than it would be in a corresponding fine search, and the value of nₘₐₓ will be smaller than in a corresponding fine search.

In one implementation, a global minimum search using method 100 with coarse search criteria is used for an initial search to find the approximate neighborhood of a global minimum. Then, following a coarse global search, a fine search using fine search criteria is used to refine the rough approximation of the global minimum obtained using the coarse global search. The fine search uses the final value of the coarse search as its starting value of the fine search.

By using a coarse global search with search criteria sufficient to find a small enough neighborhood of the global minimum that also includes local minima that are not the global minima, the fine search succeeding the coarse search does not need to be robust to the global optimization problem (i.e., a local optimization method should be adequate for the second search). Therefore, the fine search can use a local minimum optimization method and does not need to use a global optimization method, which global optimization method often converge more slowly than local optimization methods.

In one implementation, the second search can be performed using method 100. In an alternative implementation, the second search can be performed using a detector model method to find the projection lengths, such as the detector model discussed above and the detector model discussed in U.S. patent application Ser. No. 13/866,965. In another alternative implementation, iterative searches for the global minimum can be performed using different cost functions, where presumably the projection lengths corresponding to the global minimum are approximately the same for each cost function, but the projection lengths are different for purely local minima corresponding to different cost functions. Thus, finding a minimum that is simultaneously a minimum for multiple cost functions will more robustly enable the optimization to iterate to a true global minimum and avoid iterating to a purely local minimum of any one cost function. A local minimum is the smallest value of the function over a limited range, and a global minimum is the smallest value of the function over the entire range of the function.

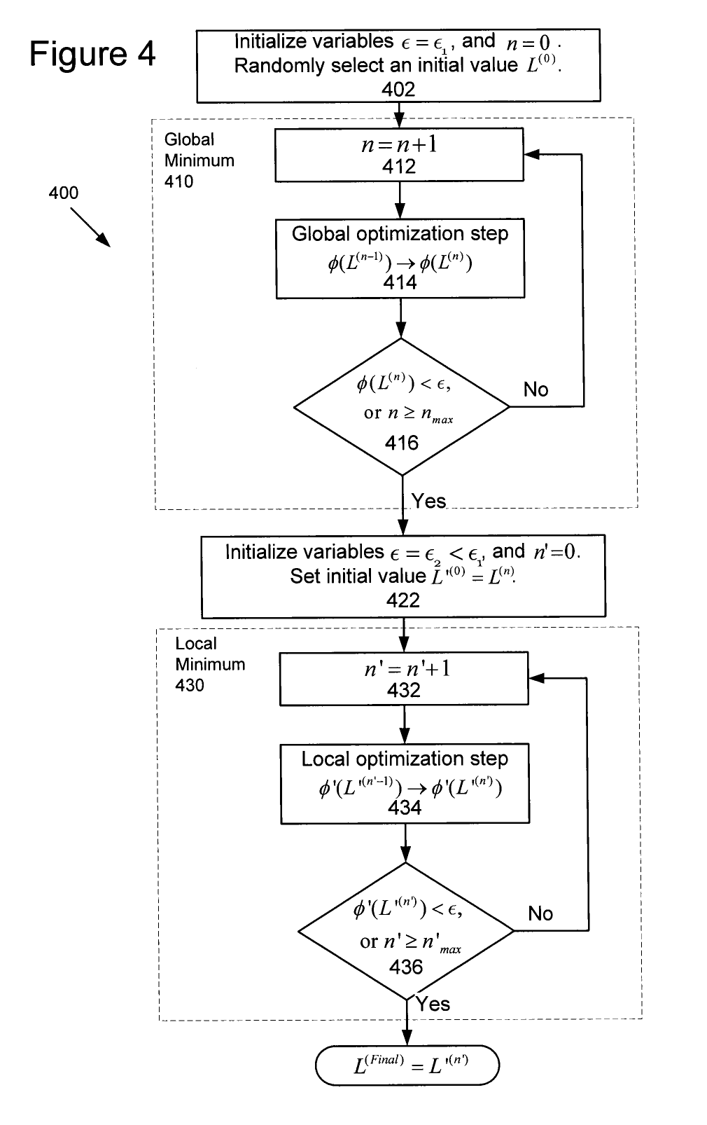

FIG. 4 shows an implementation of a two-step method 400 for obtaining optimized projection lengths by solving for the global minimum of a cost function. The global minimum process 410 is similar to the optimization process 120 shown in FIG. 2. The error limit ε, loop variable n, and initial value of the projection lengths L⁽⁰⁾ are passed in the global minimum process 410 from the initialization step 402. The loop variable n is incremented at step 412 at the beginning of each loop iteration.

Next, at step 414, a global optimization step updates the value of the projection lengths L⁽ⁿ⁾ in such a manner that the projection lengths L⁽ⁿ⁾ converge towards a global minimum of the cost function φ(L⁽ⁿ⁾). The global optimization step can be performed according to any of the global optimization methods previously discussed herein.

Next, at step 416, the convergence criteria is evaluated, and if either the cost function φ(L⁽ⁿ⁾) falls below a predefined threshold or the maximum number of iterations nₘₐₓ has been reached, then the process 410 exits the loop, returning the final value of the projection lengths L⁽ⁿ⁾.

The step 422 reinitializes the error limit ε and loop variable n′. The error limit is set to a lower value ε₂ than for the error limit ε₁ for the global minimum optimization loop. Also, in step 422 the initial values for the projection lengths are set to the final approximation found in the global minimum optimization loop, i.e., L′⁽⁰⁾=L⁽ⁿ⁾.

Next, each iteration of the local minimum loop 430 begins by incrementing the loop variable n′ at step 432.

At step 434, the projection lengths L′^{(n′)} are updated in a search for the local minimum of the cost function φ(L′^{(n′)}), where in one implementation of the method 400, the cost function φ′ used in the local minimum method 430 is different than the cost function φ used in the global minimum loop 410. In an alternative implementation of the method 400, the cost function φ′ used in the local minimum method 430 is the same as the cost function φ used in the global minimum loop 410. The method of updating the projection lengths L′^{(n′)} can correspond to any local optimization method including: a Nelder-Mead simplex method, a gradient-descent method, a Newton's method, a conjugate gradient method, a shooting method, or other known local optimization method.

At step 436, an inquiry is made as to whether the stopping criteria have been reached. FIG. 4 shows an exemplary implementation of stopping criteria, wherein if either the cost function falls below a predetermined error limit threshold, or a maximum number of loop iterations n′=n′ₘₐₓ has been reached; then the loop is exited. When the stopping criteria is satisfied, then the loop 430 is exited and the current values of the projection lengths L′^{(n′)} are output as the final projection lengths L^{(Final)}. Otherwise, the loop 430 continues until the stopping criteria are satisfied.

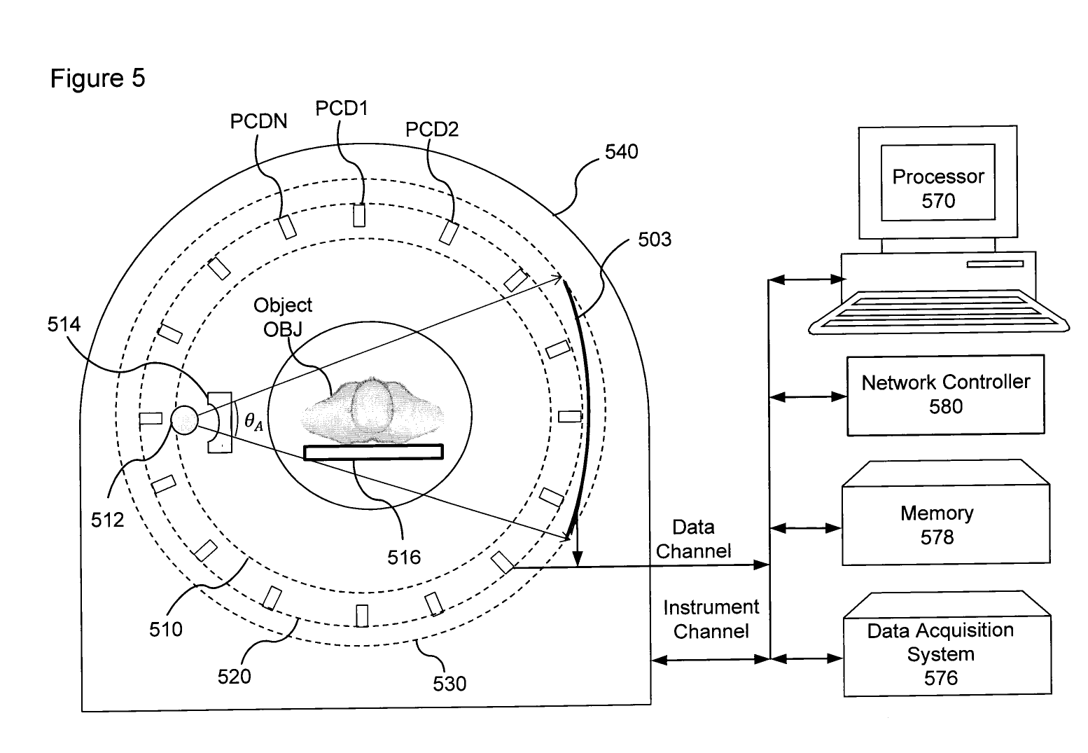

FIG. 5 shows a computed tomography (CT) scanner having both energy-integrating detectors arranged in a third-generation geometry and photon-counting detectors (PCDs) arranged in a fourth generation geometry. Illustrated in FIG. 5 is an implementation for placing the PCDs in a predetermined fourth-generation geometry in combination with a energy-integrating detector unit 503 in a predetermined third-generation geometry in a CT scanner system. The diagram illustrates relative positions among an object OBJ to be scanned resting on a table 516, an X-ray source 512, a collimator/filter 514, an X-ray detector 503, and the photon-counting detectors PCD1 through PCDN in a gantry 540. Also shown in FIG. 5 is circuitry and hardware for acquiring, storing, processing, and distributing X-ray projection data. The circuitry and hardware include: a processor 570, a network controller 580, a memory 578, and a data acquisition system 576.

In general, the photon-counting detectors PCD1 through PCDN each output a photon count for each predetermined energy bin. In addition to the sparse photon-counting detectors PCD1 through PCDN in the fourth-generation geometry, the implementation shown in FIG. 5 includes a detector unit such as the detector 503 in a conventional third-generation geometry in the CT scanner system. The detector elements in the detector unit 503 can be more densely placed along the detector unit surface than the photon-counting detectors.

In one implementation, the photon-counting detectors are sparsely placed around the object OBJ in a predetermined geometry such as a circle. For example, the photon-counting detectors PCD1 through PCDN are fixedly placed on a predetermined circular component 520 in the gantry 540. In one implementation, the photon-counting detectors PCD1 through PCDN are fixedly placed on the circular component 520 at predetermined equidistant positions. In an alternative implementation, the photon-counting detectors PCD1 through PCDN are fixedly placed on the circular component 510 at predetermined non-equidistant positions. The circular component 520 remains stationary with respect to the object OBJ and does not rotate during the data acquisition.

Each of the X-ray source 512, collimator 514, and the detector unit 503 rotate around the object OBJ while the photon-counting detectors PCD1 through PCDN are stationary with respect to the object OBJ. In one implementation, the X-ray source 512 and collimator 514 are mounted on a first rotating portion 510 such as the annular frame in the gantry 540 so that the X-ray source 512 projects X-ray radiation with a predetermined source fan beam angle θ_{A} towards the object OBJ while the X-ray source 512 rotates around the object OBJ inside the sparsely placed photon-counting detectors PCD1 through PCDN. Furthermore, a detector unit 503 is mounted on a second rotating portion 530. The rotating portion 530 mounts the detector unit 503 at a diametrically opposed position from the X-ray source 512 across the object OBJ and rotates outside the stationary circular component 520.

In one implementation, the X-ray source 512 optionally travels a helical path relative to the object OBJ, which is moved in a predetermined direction that is perpendicular to the rotational plane of the rotating portion 510.

As the X-ray source 512 and the detector unit 503 rotate around the object OBJ, the photon-counting detectors PCDs and the detector unit 503 respectively detect the transmitted X-ray radiation during data acquisition. The photon-counting detectors PCD1 through PCDN intermittently detect the X-ray radiation that has been transmitted through the object OBJ and individually output a count value representing a number of photons, for each of predetermined energy bins. On the other hand, the detector elements in the detector unit 503 continuously detect the X-ray radiation that has been transmitted through the object OBJ and output the detected signals as the detector unit 503 rotates. In one implementation, the detector unit 503 has densely placed energy-integrating detectors in predetermined channel and segment directions on the detector unit surface.

In one implementation, the X-ray source 512, the photon-counting detectors and the detector unit 503 collectively form three predetermined circular paths that differ in radius. The photon-counting detectors are sparsely placed along a first circular path around the object OBJ while at least one X-ray source 512 rotates along a second circular path around the object OBJ. Further, the detector unit 503 travels along a third circular path. The above exemplary embodiment illustrates that the third circular path is the largest and outside the first and second circular paths around the object OBJ. Although not illustrated, an alternative embodiment optionally changes the relative relation of the first and second circular paths so that the second circular path for the X-ray source 512 is larger and outside the first circular path of the sparsely placed photon-counting detectors PCD1 through PCDN around the object OBJ. Furthermore, in another alternative embodiment, the X-ray source 512 also optionally travels on the same third circular path as the detector unit 503.

There are other alternative embodiments for placing the photon-counting detectors in a predetermined fourth-generation geometry in combination with the detector unit in a predetermined third-generation geometry in the CT scanner. Several alternative embodiments of the X-ray CT Scanner as described in U.S. Patent Publication No. 2013/0251097 A1, herein incorporated by reference in its entirety. Additional embodiments of the X-ray CT Scanner are also described in U.S. patent application Ser. No. 14/092,998, herein incorporated by reference in its entirety.

In one alternative implementation, the detector unit 503 is not present and the only detectors are the photon counting detectors.

In one implementation, the X-ray source 512, which is configured to perform a kV-switching function for emitting X-ray radiation at a predetermined high-level energy and at a predetermined low-level energy. In still another alternative embodiment, the X-ray source 512 is a single source emitting a broad spectrum of X-ray energies. In still another embodiment, the X-ray source 512 is more than a single X-ray emitter and each emitter can emit X-rays separately and emits a different spectrum of X-ray energies.

The detector unit 503 can use energy integrating detectors such as scintillation elements with photo-multiplier tubes or avalanche photo-diodes to detect the resultant scintillation photons from scintillation events resulting from the X-ray radiation interacting with the scintillator elements. The scintillator elements can include a crystalline scintillating material (e.g., NaI(Tl), CsI(Tl), CsI(Na), CsI(pure), CsF, KI(Tl), LiI(Eu), BaF₂, CaF₂(Eu), ZnS(Ag), CaWO₄, CdWO₄, YAG(Ce), Y₃Al₅O₁₂(Ce), GSO, LSO, LaCl₃(Ce), LaBr₃(Ce), LYSO, BGO, LaCl₃(Ce), LaBr₃(Ce), C₁₄H₁₀, C₁₄H₁₂, and C₁₀H₈), an organic liquid scintillating material (e.g., an organic solvent with a fluor such as p-terphenyl (C₁₈H₁₄), PBD (C₂₀H₁₄N₂O), butyl PBD (C₂₄H₂₂N₂O), or PPO (C₁₅H₁₁NO)), a plastic scintillating material (e.g., a flour suspended in a solid polymer matrix), or other know scintillating materials.

The photon counting detectors can use a direct X-ray radiation detectors based on semiconductors, such as cadmium telluride (CdTe), cadmium zinc telluride (CZT), silicon (Si), mercuric iodide (HgI₂), and gallium arsenide (GaAs). These direct X-ray detectors have much faster time response than indirect detectors. The fast time response of direct detectors enables them to resolve individual X-ray detection events with only limited pile-up even at the high X-ray fluxes typical of clinical X-ray imaging applications. The amount of energy of the X-ray detected is proportional to the signal generated at the direct detector, and the energies of detection events can be binned into a discrete number of corresponding energy bins yielding spectrally resolved X-ray projection measurements.

The CT scanner also includes a data channel that routes projection measurement results from the photon counting detectors and the detector unit 503 to a data acquisition system 576, a processor 570, memory 578, network controller 580. The data acquisition system 576 controls the acquisition, digitization, and routing of projection data from the detectors. The data acquisition system 576 also includes radiography control circuitry to control the rotation of the annular rotating frames 510 and 530. In one implementation data acquisition system 576 will also control the movement of the bed 516, the operation of the X-ray source 512 (e.g., the high voltage supplied to the X-ray source), and the operation of the X-ray detectors (e.g., gating of the X-ray detectors and their read out). The data acquisition system 576 can be a centralized system or alternatively it can be a distributed system. In an implementation, the data acquisition system 576 is integrated with the processor 570. The processor 570 performs functions including reconstructing images from the projection data, pre-reconstruction processing of the projection data, and post-reconstruction processing of the image data. The pre-reconstruction processing of the projection data can include correcting for detector calibrations, detector nonlinearities, polar effects, noise balancing, and material decomposition. Post-reconstruction processing can include filtering and smoothing the image, volume rendering processing, and image difference processing as needed. The image reconstruction process can be performed using filtered back projection, iterative image reconstruction methods, or stochastic image reconstruction methods. Both the processor 570 and the data acquisition system 576 can make use of the memory 578 to store, e.g., projection data, reconstructed images, calibration data and parameters, and computer programs.

The processor 570 can include a CPU that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Alternatively, the CPU in the reconstruction processor may execute a computer program including a set of computer-readable instructions that perform the functions described herein, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xenon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

In one implementation, the reconstructed images can be displayed on a display. The display can be an LCD display, CRT display, plasma display, OLED, LED or any other display known in the art.

The memory 578 can be a hard disk drive, CD-ROM drive, DVD drive, FLASH drive, RAM, ROM or any other electronic storage known in the art.

The network controller 580, such as an Intel Ethernet PRO network interface card from Intel Corporation of America, can interface between the various parts of the CT scanner. Additionally, the network controller 580 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G and 4G wireless cellular systems. The wireless network can also be WiFi, Bluetooth, or any other wireless form of communication that is known.

As will be further illustrated the above described implementations are examples and additional implementation can vary from the above examples in many aspects. For example, although certain spatial relationships of the trajectories or paths are disclosed among the source 512, the PCDs and the energy integrating detector 503, the spatial relationship is relative and not limited to a particular relation as illustrated in FIG. 5. Another example is that the energy differentiating detectors PCD are mounted inside the gantry 540 in the implementation shown in FIG. 5 while the PCDs of another implementation are initially mounted in a retrofitting unit or device that is not illustrated in FIG. 5 before the retrofitting device is placed in an existing CT scanner system. Lastly, although a single pair of the energy integrating detector 503 and the radiation source 512 is illustrated in the implementation shown in FIG. 5, an additional pair of the energy integrating detector 503 and the radiation source 512 is incorporated in another implementation according to the current invention.

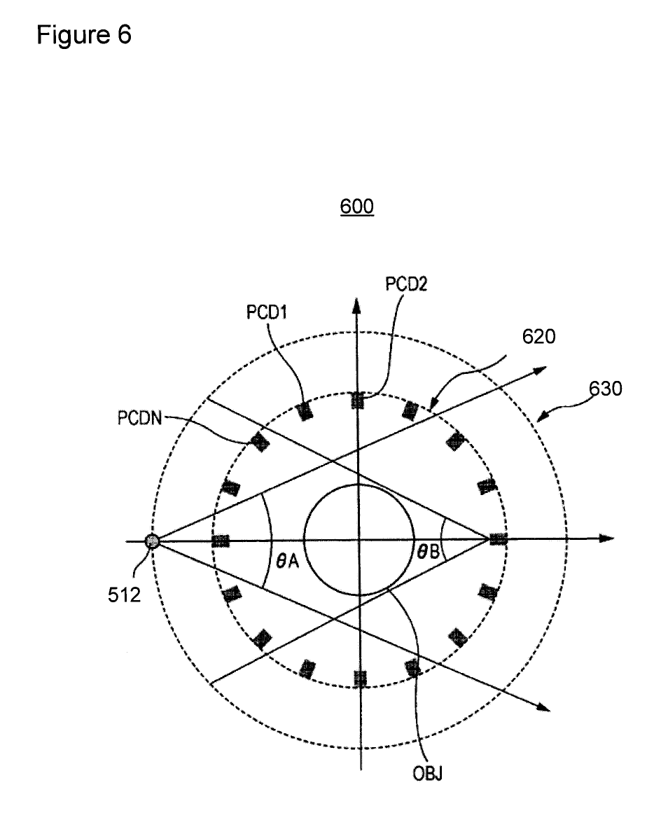

Now referring to FIG. 6, a diagram illustrates one implementation for placing the PCDs in a predetermined fourth-generation geometry in the CT scanner system according to the current invention. The diagram merely illustrates a relative relationship among an object or the object OBJ to be scanned, an X-ray source or radiation emitting source 512 and photon counting detectors PCD1 through PCDN in one exemplary implementation. For the sake of simplicity, the diagram excludes other components and units that may be necessary in acquiring and processing data as well as reconstructing an image based upon the acquired data. In general, the photon counting detectors PCD1 through PCDN are made of a device and output a photon count for each of predetermined energy components. Although approximately one hundred to three hundred photon counting detectors are utilized in certain implementations, the above numerical range of the photon counting detectors is merely exemplary, and the claimed invention is not necessarily limited to any particular number of the photon counting detectors.

Still referring to FIG. 6, one implementation includes a predetermined number of the PCDs, which are sparsely placed around the object OBJ in a predetermined geometry such as a circle. For example, the photon counting detectors PCD1 through PCDN are fixedly placed on a predetermined circular component 620 in the gantry 600. Furthermore, the photon counting detectors PCD1 through PCDN are fixedly placed on the circular component 620 at predetermined equidistant positions in one implementation. In another implementation, the photon counting detectors PCD1 through PCDN are fixedly placed on the circular component 620 at predetermined non-equidistant positions. The circular component 620 remains stationary with respect to the object OBJ and fails to rotate during the data acquisition. On the other hand, the X-ray source 512 is located outside the circular component 620 and is mounted on a rotating portion 630 such as the annular frame in the gantry 600 so that the X-ray source 512 projects X-ray with a predetermined source fan beam angle θ_{A} towards the object OBJ while the X-ray source 512 rotates around the object OBJ outside the sparsely placed photon counting detectors PCD1 through PCDN. Consequently, the photon counting detectors PCD1 through PCDN individually detect with a predetermined detector fan beam angle θ_{B} the X-ray that has been transmitted through the object OBJ and output a number of photons for each of predetermined energy components.

In certain implementations, the energy differentiating detectors PCD1 through PCDN are initially housed in the module housing of a modular retrofitting unit before the retrofitting device is placed in an existing CT scanner system. The above described modular retrofitting device is optionally used with other implementations. That is, the modular device 620 with the energy differentiating detectors is retrofitted in an existing image scanner for reconstructing an image. The image scanner rotates a radiation emitting source along a first path around a predetermined center while continuously emits energy towards an object. The image scanner optionally rotates an energy integrating detector for detecting intensity data along a second path around the predetermined center. The modular device further includes a predetermined number of energy differentiating detectors for detecting spectral data and a module housing for housing a predetermined number of the energy differentiating detectors that are fixedly placed along a third path, third path being inside the first path as the module housing is retrofitted into the existing image scanner, whereas the scanner reconstructs an image based upon the intensity data and the spectral data. The above described paths include certain predetermined trajectories such as a circumference, a helix and a polygon, but are not limited to a particular set of predetermined paths in a predetermined combination. Furthermore, the size of the modular retrofitting unit or device 620 is not necessarily limited to a gantry or a housing of the existing CT scanner system. The modular retrofitting unit or device 620 is also optionally attached to a gantry or a housing of the existing CT scanner system in a detachable or fixed manner.

FIG. 6 also discloses that the X-ray from the source 512 travels through openings or gaps between the sparsely placed photon counting detectors PCD1 through PCDN towards the object OBJ. Some portion of the emitted X-ray is blocked by certain ones of the sparsely placed photon counting detectors PCD1 through PCDN depending upon an angle with respect to the source 512. In other words, a certain portion of the emitted X-ray projects onto the back surface of some of the sparsely placed photon counting detectors PCD1 through PCDN at any given time as the source 512 is rotated around the predetermined trajectory 630. The remaining X-ray travels through the gap and reaches certain ones of the photon counting detectors PCD1 through PCDN, whose detecting surface is facing the source 512 and is substantially within the predetermined source fan beam angle θ_{A}, and each of these photon counting detectors PCD1 through PCDN individually detect with the predetermined detector fan beam angle θ_{B}.

In the above implementation, the PCDs are sparsely and fixedly placed along a first circular path around the object OBJ while at least one X-ray source 512 rotates along a second circular path around the object OBJ. Furthermore, the above implementation illustrates that the first circular path is smaller and inside the second circular path around the object OBJ. There are other alternative implementations for placing the PCDs in a predetermined fourth-generation geometry in the CT scanner system according to the current invention. Although it is not illustrated in a drawing, an alternative implementation optionally includes a first path that is substantially circular and also a non-circular first path such as a predetermined polygon along which the photon counting detectors PCD1 through PCDN are sparsely placed.

Again, although it is not illustrated in a drawing, an alternative implementation optionally includes more than one X-ray source 512, and a plurality of the X-ray sources 512 is mounted on the rotating portion 630 such as the annular frame at a predetermined angle with each other. At least one of the X-ray sources 512 is optionally a single energy source in certain implementations. By the same token, a second alternative implementation optionally includes the X-ray source 512, which is configured to perform a kV-switching function for emitting X-ray at a predetermined high-level energy and a predetermined low-level energy. Furthermore, the radiation emitting source or the X-ray source 512 optionally modulates a combination of a radiation energy level and an intensity level over time.

The above implementation according to the current invention also provides a protective rear cover for each of the photon counting detectors PCD1 through PCDN that are irradiated from behind in a short distance. As the X-ray source 512 travels outside the first circular path of the sparsely placed photon counting detectors PCD1 through PCDN, the photon counting detectors PCD1 through PCDN′ are protected by the protective layer from the X-ray irradiation on the rear surface in order to substantially reduce undesirable effects.

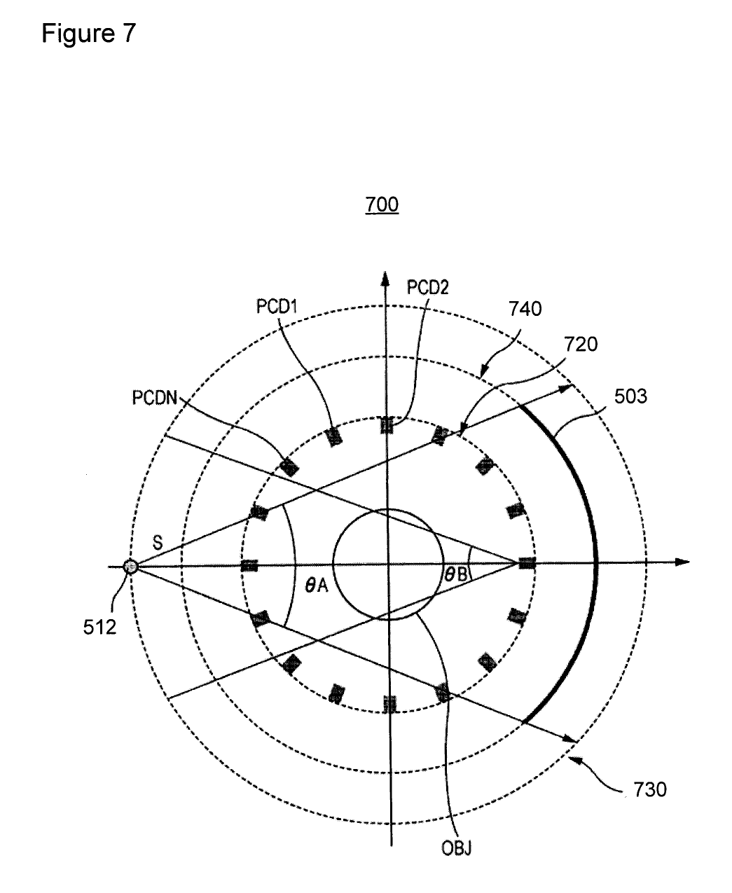

Now referring to FIG. 7, a diagram illustrates another implementation for placing the PCDs in a predetermined fourth-generation geometry in combination with a detector unit in a predetermined third generation geometry in the CT scanner system according to the current invention. The diagram merely illustrates a relative relationship among an object OBJ to be scanned, an X-ray source or radiation emitting source 512, an energy integrating detector 503 and the energy differentiating detectors PCD1 through PCDN in one exemplary implementation. For the sake of simplicity, the diagram excludes other components and units that may be necessary in acquiring and processing data as well as reconstructing an image based upon the acquired data.

The implementation utilizes a combination of the two types of detectors. In general, the photon counting detectors PCD1 through PCDN are made from a device and output a photon count for each of predetermined energy components. Although approximately one hundred to three hundred photon counting detectors are utilized in certain implementations, the above numerical range of the photon counting detectors is merely exemplary, and the claimed invention is not necessarily limited to any particular number of the photon counting detectors. In addition to the sparsely placed photon counting detectors PCD1 through PCDN in the fourth-generation geometry, the implementation of FIG. 7 now further includes an additional detector unit such as the energy integrating detector 503 in a third-generation geometry in the CT scanner system according to the current invention. The detector elements in the detector unit 503 are generally more densely placed along the detector unit surface than the PCDs in the exemplary implementation. The detector surface of the detector unit 503 is optionally flexible, cylinder centered at iso-center at the source, sphere centered at the source or a flat panel.

Still referring to FIG. 7, one implementation includes a predetermined number of the PCDs, which are sparsely placed around the object OBJ in a predetermined geometry such as a circle. For example, the photon counting detectors PCD1 through PCDN are fixedly placed on a predetermined circular component 720 in the gantry 700. Furthermore, the photon counting detectors PCD1 through PCDN are fixedly placed on the circular component 720 at predetermined equidistant positions in one implementation. In another implementation, the photon counting detectors PCD1 through PCDN are fixedly placed on the circular component 720 at predetermined non-equidistant positions. The circular component 720 remains stationary with respect to the object OBJ and fails to rotate during the data acquisition. The circular component 720 also provides a gap between the two adjacent ones of the photon counting detectors PCD1 through PCDN, and these gaps allows the transmission of the X-ray without substantial interference. Although it is not illustrated in a drawing, an alternative implementation optionally includes a predetermined component 720 that is substantially circular and non-circular such as polygonal along which the photon counting detectors PCD1 through PCDN are sparsely placed.

Both the X-ray source 512 and the detector unit 503 rotate around the object OBJ while the photon counting detectors PCD1 through PCDN remain stationary with respect to the object OBJ. In one exemplary implementation, the X-ray source 512 is mounted on a first rotating portion 730 such as the annular frame in the gantry 700 so that the X-ray source 512 projects X-ray with a predetermined source fan beam angle θ_{A} towards the object OBJ while the X-ray source 512 rotates around the object OBJ outside the sparsely placed photon counting detectors PCD1 through PCDN. Furthermore, an additional detector unit 503 is mounted on a second rotating portion 740 in the third-generation geometry in the above exemplary implementation of the CT scanner system according to the current invention. The rotating portion 740 mounts the detector unit 503 at a diametrically opposed position from the X-ray source 512 across the object OBJ and rotates outside the stationary circular component 720, on which the photon counting detectors PCD1 through PCDN are fixedly placed in a predetermined sparse manner.

In one implementation, the rotating portions 730 and 740 are integrally constructed as a single component such as the annular frame 102 to maintain the 180-degree angle between the X-ray source 512 and the detector unit 503 as they rotate about the object OBJ with a different radius. In an optional implementation, the rotating portions 730 and 740 are separate components but synchronously rotate to maintain the X-ray source 512 and the detector unit 503 in the fixedly opposed positions at 180 degrees across the object OBJ. Furthermore, the X-ray source 512 optionally travels a helical path as the object is moved in a predetermined direction that is perpendicular to the rotational plane of the rotating portion 730. Although it is not illustrated in the diagram, the rotating portions 730 and 740 are reversed in their diameters in another alternative implementation. That is, although the source 512 and the detector unit 503 travel outside the sparsely placed photon counting detectors PCD1 through PCDN, the source 512 has a trajectory that is inside that of the detector unit 503 in the alternative implementation while they travel at a diametrically fixed position with each other.

In the above exemplary implementation, the X-ray source 512, the photon counting detectors (PCD) and the detector unit 503 collectively form three predetermined circular paths that differ in radius. The PCDs are sparsely placed along a first circular path around the object OBJ while at least one X-ray source 512 rotates along a second circular path around the object OBJ. Further, the detector unit 503 travels along a third circular path. The above exemplary implementation illustrates that the second circular path is the largest and outside the first and third circular paths around the object OBJ. Although it is not illustrated in, yet another alternative implementation optionally changes the X-ray source 512 to travel on the same third circular path as the detector unit 503. There are other alternative implementations for placing the PCDs in a predetermined fourth-generation geometry in combination with the detector unit in a predetermined third-generation geometry in the CT scanner system according to the current invention. The X-ray source 512 is optionally a single energy source in certain implementations. By the same token, an additional alternative implementation optionally includes the X-ray source 512, which is configured to perform a kV-switching function for emitting X-ray at a predetermined high-level energy and a predetermined low-level energy. Furthermore, the radiation emitting source or the X-ray source 512 optionally modulates a combination of a radiation energy level and an intensity level over time.

As the X-ray source 512 and the detector unit 503 rotate around the object OBJ, the photon counting detectors PCDs and the detector unit 503 respectively detect the transmitted X-ray during the data acquisition. The photon counting detectors PCD1 through PCDN intermittently detect with a predetermined detector fan beam angle θ_{B} the X-ray that has been transmitted through the object OBJ and individually output a number of photons for each of predetermined energy components. On the other hand, the detector elements in the detector unit 503 continuously detect the X-ray that has been transmitted through the object OBJ and output the detected energy integration signals as the detector unit 503 rotates. Although the additional characteristics of the detector elements in the detector unit 503 will be later described in details, one implementation of the detector unit 503 has densely placed energy integrating detectors in a predetermined channel and segment directions on the detector unit surface.

FIG. 7 further discloses that since the source 512 travels outside the photon counting detectors PCD1 through PCDN, the X-ray is projected through openings or gaps between the sparsely placed photon counting detectors PCD1 through PCDN towards the object OBJ. Some portion of the emitted X-ray is blocked by certain ones of the sparsely placed photon counting detectors PCD1 through PCDN depending upon an angle with respect to the source 512. In other words, a certain portion of the emitted X-ray projects onto the back surface of some of the sparsely placed photon counting detectors PCD1 through PCDN at any given time as the source 512 is rotated around the predetermined trajectory 730. The remaining X-ray travels through the gap and reaches certain ones of the photon counting detectors PCD1 through PCDN, whose detecting surface is facing the source 512 and is substantially within the predetermined source fan beam angle θ_{A}. Each of these photon counting detectors PCD1 through PCDN individually detects with the predetermined detector fan beam angle θ_{B}. Furthermore, still some of the remaining X-ray travel an additional distance through another gap between certain ones of the photon counting detectors PCD1 through PCDN and reach the detector unit 503, w hose detecting surface is substantially within the predetermined source fan beam angle θ_{A}.

The above implementations according to the current invention also provide a protective rear cover for each of the photon counting detectors PCD1 through PCDN that are irradiated from behind in a short distance. As the X-ray source 512 travels outside the first circular path of the sparsely placed photon counting detectors PCD1 through PCDN, the photon counting detectors PCD1 through PCDN are protected by the protective layer from the X-ray irradiation on the rear surface in order to substantially reduce undesirable effects.

In general, the photon counting detectors PCD1 through PCDN are sparsely positioned along the circular component 720. Although the photon counting detectors PCD1 through PCDN acquire sparse view projection data, the acquired projection data is sufficient for at least dual energy reconstruction with a certain sparse view reconstruction technique. In addition, the detector unit 503 also acquires another set of projection data, and the projection data from the detector unit 503 is used to generally improve image quality. In case that the detector unit 503 consists of energy integrating detectors with anti-scatter grids, the projection data from the detector unit 503 is used to correct scatter on the projection data from the PCDs. In the above alternative implementations, the integrating detectors optionally need to be calibrated in view of X-ray transmission through the predetermined circular component 720 and some of the PCDs. In acquiring the projection data, a sampling on the source trajectory is optionally made dense in order to enhance spatial resolution.

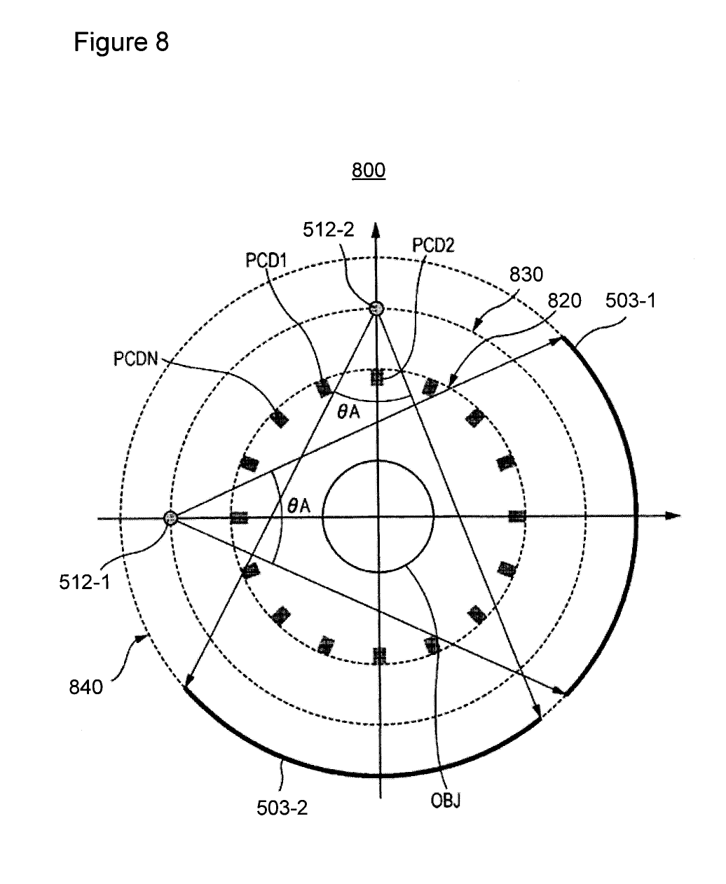

Now referring to FIG. 8, a diagram illustrates another implementation for placing the PCDs in a predetermined fourth-generation geometry in combination with two X-ray sources and two detector units in a predetermined third-generation geometry in the CT scanner system according to the current invention. The diagram merely illustrates a relative relationship among an object OBJ to be scanned, two radiation emitting sources or X-ray sources 512-1 and 512-2, two X-ray detector units 503-1 and 503-2 and the photon counting detectors PCD1 through PCDN in one exemplary implementation. For the sake of simplicity, the diagram excludes other components and units that are necessary in acquiring and processing data as well as reconstructing an image based upon the acquired data.

As already described, approximately one hundred to three hundred photon counting detectors PCD1 through PCDN are generally utilized in certain implementations. However, the above numerical range of the photon counting detectors is merely exemplary, and the claimed invention is not necessarily limited to any particular number of the photon counting detectors. In addition to the sparse photon counting detectors PCD1 through PCDN in the fourth-generation geometry, the exemplary implementation of FIG. 8 now further includes at least two detector units 503-1 and 503-2 in a predetermined third generation geometry in the CT scanner system according to the current invention. Although the detector units 503-1 and 503-2 are both energy integrating detectors in the implementation, the two detectors are optionally different in other implementations.

Still referring to FIG. 8, one implementation includes a predetermined number of the PCDs, which are sparsely placed around the object OBJ in a predetermined geometry such as a circle. For example, the photon counting detectors PCD1 through PCDN are fixedly placed on a predetermined circular component 820 in the gantry 800. Furthermore, the photon counting detectors PCD1 through PCDN are fixedly placed on the circular component 820 at predetermined equidistant positions in one implementation. In another implementation, the photon counting detectors PCD1 through PCDN are fixedly placed on the circular component 820 at predetermined non-equidistant positions. The circular component 820 remains stationary with respect to the object OBJ and fails to rotate during the data acquisition. The circular component 820 also provides a gap between the two adjacent ones of the photon counting detectors PCD1 through PCDN, and these gaps allows the transmission of the X-ray without substantial interference. Although it is not illustrated in a drawing, an alternative implementation optionally includes a predetermined component 820 that is substantially circular and non-circular such as polygonal along which the photon counting detectors PCD1 through PCDN are sparsely placed.

The two pairs of the X-ray sources 512-1, 1 01-2 and the detector units 503-1, 503-2 rotate around the object OBJ while the photon counting detectors PCD1 through PCDN remain stationary with respect to the object OBJ. For each pair, a rotating portion 840 respectively mounts the detector units 503-1 and 503-2 at a diametrically opposed position from the X-ray sources 512-1 and 512-2 across the object OBJ and rotates outside the stationary circular component 820, on which the photon counting detectors PCD1 through PCDN are fixedly placed in a predetermined sparse manner. Furthermore, a first pair of the X-ray source 512-1 and the detector unit 503-1 is mounted in a substantially perpendicular manner with respect to a second pair of the X-ray source 512-2 and the detector unit 503-2 in the gantry 800 in the above exemplary implementation. Each of the X-ray sources 512-1 and 512-2 projects X-ray with a predetermined source fan beam angle θ_{A} towards the object OBJ while the X-ray sources 512-1 and 512-2 rotate around the object OBJ outside the sparsely placed photon counting detectors PCD1 through PCDN.

In one implementation, the rotating portions 830 and 840 are integrally constructed as a single component such as the annular frame 102 to maintain the 180-degree angle between the X-ray sources 512-1, 1 01-2 and the detector units 503-1, 103-2 as they rotate about the object OBJ with a different radius. In an optional implementation, the rotating portions 830 and 840 are separate components but synchronously rotate to maintain the X-ray sources 512-1, 512-2 and the detector units 503-1, 503-2 in the fixedly opposed positions at 180 degrees across the object OBJ. Furthermore, the X-ray sources 512-1 and 512-2 optionally travel a helical path as the object is moved in a predetermined direction that is perpendicular to the rotational plane of the rotating portion 830. Although it is not illustrated in the diagram, the rotating portions 830 and 840 are reversed in their diameter in another alternative embodiment. That is, although the sources 512-1, 512-2 and the detector units 503-1 and 503-2 travel outside the sparsely placed photon counting detectors PCD1 through PCDN, the sources 512-1, 1 01-2 have a trajectory that is outside that of the detector units 503-1 and 503-2 while they travel at a diametrically fixed position with each other.

In the above exemplary implementation, the X-ray sources 512-1, 1 01-2, the photon counting detectors (PCD) and the detector units 503-1, 503-2 collectively form three predetermined circular paths that differ in radius. The PCDs are sparsely placed along a first circular path around the object OBJ while the X-ray sources 512-1 and 512-2 rotate along a second circular path around the object OBJ. Further, the detector units 503-1 and 503-2 both travel along a third circular path. The above exemplary implementation illustrates that the third circular path is the largest and outside the first and second circular paths around the object OBJ. Although it is not illustrated in a drawing, yet another alternative implementation optionally changes the X-ray sources 512-1 and 512-2 to travel on the same third circular path as the detector units 503-1 and 503-2.

There are other alternative implementations for placing the PCDs in a predetermined fourth-generation geometry in combination with two sources and two detector units in a predetermined third-generation geometry in the CT scanner system according to the current invention. At least one of the X-ray sources 512-1 and 512-2 is optionally a single energy source in certain implementations. By the same token, an additional alternative implementation optionally includes the X-ray sources 512-1 and or 512-2, which are configured to perform a kV-switching function for emitting X-ray at a predetermined high-level energy and a predetermined low-level energy. Furthermore, at least one of the radiation emitting sources or the X-ray sources 512-1 and 512-2 optionally modulates a combination of a radiation energy level and an intensity level over time.

As the X-ray sources 512-1, 512-2 and the detector units 503-1, 503-2 rotate around the object OBJ, the PCDs and the detector units 503-1, 503-2 respectively detect the transmitted X-ray during the data acquisition. The photon counting detectors PCD1 through PCDN intermittently detect with a predetermined detector fan beam angle θ_{B} the X-ray that has been transmitted through the object OBJ and individually output a number of photons for each of predetermined energy components. On the other hand, the detector elements in the detector units 503-1 and 503-2 continuously detect the X-ray that has been transmitted through the object OBJ and output the detected energy integration signals as the detector units 503-1 and 503-2 rotate. Although the additional characteristics of the detector elements in the detector units 503-1 and 503-2 will be later described in details, one implementation of the detector units 503-1 and 503-2 has densely placed energy integrating detectors in a predetermined channel and segment directions on the detector unit surface.

FIG. 8 further discloses that since the X-ray sources 512-1 and 512-2 travel outside the photon counting detectors PCD1 through PCDN, the X-ray is projected through openings or gaps between the sparsely placed photon counting detectors PCD1 through PCDN towards the object OBJ. Some portion of the emitted X-ray is blocked by certain ones of the sparsely placed photon counting detectors PCD1 through PCDN depending upon an angle with respect to the X-ray sources 512-1 and 512-2. In other words, a certain portion of the emitted X-ray projects onto the back surface of some of the sparsely placed photon counting detectors PCD1 through PCDN at any given time as the X-ray sources 512-1 and 512-2 are rotated around the predetermined trajectory 830. The remaining X-ray travels through the gap and reaches certain ones of the photon counting detectors PCD1 through PCDN, whose detecting surface is facing the source 512-1 or 512-2 and is substantially within the predetermined source fan beam angle θ_{A}. Each of these photon counting detectors PCD1 through PCDN individually detects with the predetermined detector fan beam angle θ_{B}. Furthermore, still some of the remaining X-ray travel an additional distance through another gap between certain ones of the photon counting detectors PCD1 through PCDN and reach the detector unit 503-1 or 503-2, whose detecting surface is substantially within the predetermined source fan beam angle θ_{A}.

The above implementations according to the current invention also provide a protective rear cover for each of the PCDs that are irradiated from behind in a short distance. As the X-ray sources 512-1 and 512-2 travel outside the first circular path of the sparsely placed photon counting detectors PCD1 through PCDN, the photon counting detectors PCD1 through PCDN are protected by the protective layer from the X-ray irradiation on the resurface in order to substantially reduce undesirable effects.

In general, the photon counting detectors PCD1 through PCDN are sparsely positioned along the circular component 820. Although the photon counting detectors PCD1 through PCDN acquire sparse view projection data, the acquired projection data is sufficient for at least dual energy reconstruction with a certain sparse view reconstruction technique. In addition, the detector units 503-1 and 503-2 respectively acquire another set of projection data, and the projection data from the detector units 503-1 and 503-2 is used to generally improve image quality. In case that the detector units 503-1 and 503-2 consist of integrating detectors with anti-scatter grids, the projection data from the detector units 503-1 and 503-2 is used to correct scatter on the projection data from the PCDs. In the above alternative implementations, the integrating detectors optionally need to be calibrated in view of X-ray transmission through the predetermined circular component 820 and some of the PCDs. In acquiring the projection data, a sampling on the source trajectory is optionally made dense in order to enhance spatial resolution.

While certain implementations have been described, these implementations have been presented by way of example only, and are not intended to limit the teachings of this disclosure. Indeed, the novel methods, apparatuses and systems described herein may be embodied in a variety of other forms; furthermore, various omissions, substitutions and changes in the form of the methods, apparatuses and systems described herein may be made without departing from the spirit of this disclosure.

1. An apparatus, comprising:
processing circuitry configured to
obtain projection data having a plurality of energy components, wherein the projection data represents an intensity of radiation having been transmitted through an imaged object and then detected at a plurality of detector elements;
calculate a cost function representing differences between the projection data and calculated data over the plurality of energy components, wherein the calculated data represents intensity of radiation transmitted through the imaged object, the calculated data being calculated using a detector model that approximates attenuation of the radiation, the detector model using a plurality of projection lengths, with each projection length corresponding to a respective material model of a plurality of material models; and
optimize the plurality of projection lengths until the calculated cost function converges to approximate a global minimum of the cost function.

2. The apparatus according to claim 1, wherein the processing circuitry is further configured to
optimize the plurality of projection lengths using a stochastic optimization method that is one of a genetic algorithm, a simulated annealing method, a quantum annealing method, a swarm algorithm, an evolutionary algorithm, a random search, a replica exchange method, and a reactive search optimization method.

3. The apparatus according to claim 1, wherein the processing circuitry is further configured to optimize the plurality of projection lengths by
choosing a plurality of random projection lengths, the random projection lengths being chosen to be within a sample space surrounding a plurality of current projection lengths;
assigning the plurality of current projection lengths to be equal to the plurality of random projection lengths, when the cost function corresponding to the plurality of random projection lengths is less than the cost function corresponding to the plurality of current projection lengths;
maintaining the plurality of current projection lengths unchanged, when the cost function corresponding to the plurality of random projection lengths is greater than or equal to the cost function corresponding to the plurality of current projection lengths; and
repeating the steps of choosing the plurality of random projection lengths, assigning the plurality of current projection lengths, and maintaining the plurality of current projection lengths, until either the cost function corresponding to the plurality of current projection lengths is less than a predetermined threshold or a number of iterations reaches a predetermined maximum number of iterations.

4. The apparatus according to claim 1, wherein the processing circuitry is further configured to calculate the cost function using a method that is one of a least squares difference between the projection data and calculated data method, a weighted least squares difference between the projection data and calculated data method, and a Poisson likelihood function method.

5. The apparatus according to claim 4, wherein the processing circuitry is further configured to calculate the cost function using the detector model having a linear detector response term and a nonlinear detector response term, wherein the linear and nonlinear detector response terms each include a detector dead time and a radiation flux.

6. The apparatus according to claim 5, wherein the processing circuitry is further configured to calculate the cost function using the radiation flux determined using a reference intensity representing a radiation intensity of the radiation at a radiation source.

7. The apparatus according to claim 1, wherein the processing circuitry is further configured to optimize the plurality of projection lengths according to a multi-step optimization method having a first step and a second step, wherein
the first step includes performing a global optimization method solving for the plurality of projection lengths that minimize a first cost function; and
the second step includes using the optimized plurality of projection lengths obtained in the first step to perform a second optimization method solving for the plurality of projection lengths that minimize a second cost function.

8. The apparatus according to claim 7, wherein the processing circuitry is further configured to optimize the plurality of projection lengths, wherein the first step includes performing a coarse optimization and the second step includes performing a fine optimization.

9. The apparatus according to claim 7, wherein the processing circuitry is further configured to optimize the plurality of projection lengths, wherein the first step includes performing a global optimization and the second step includes performing a local optimization.

10. An apparatus, comprising:
an X-ray source radiating X-rays;
a plurality of detector elements each configured to detect a plurality of energy components of the X-rays that are radiated from the X-ray source and generate projection data; and
processing circuitry configured to
obtain the projection data having the plurality of energy components, wherein the projection data represents an intensity of radiation having been transmitted through an imaged object and then detected at the plurality of detector elements,
calculate a cost function representing differences between the projection data and calculated data over the plurality of energy components, wherein the calculated data represents intensity of radiation transmitted through the imaged object, the calculated data being calculated using a detector model that approximates attenuation of the radiation, the detector model using a plurality of projection lengths, with each projection length corresponding to a respective material model of a plurality of material models, and
optimize the plurality of projection lengths until the calculated cost function converges to approximate a global minimum of the cost function.

11. The apparatus according to claim 10, further comprising:
a reference detector configured to detect a reference X-ray intensity I_{ref} emitted at the X-ray source,
wherein the processing circuitry is further configured to calculate an incident X-ray flux of each respective energy component of each respective detector element as the product of the reference X-ray intensity with a corresponding predetermined flux calibration factor.

12. The apparatus according to claim 1, wherein the processing circuitry is further configured to reconstruct a plurality of images using the optimized plurality of projection lengths, wherein each reconstructed image corresponding to a respective material model.

13. A method, comprising:
obtaining projection data having a plurality of energy components, wherein the projection data represents an intensity of radiation having been transmitted through an imaged object and then detected at a plurality of detector elements;
calculating a cost function representing differences between the projection data and calculated data over the plurality of energy components, wherein the calculated data represents intensity of radiation transmitted through the imaged object, the calculated data being calculated using a detector model that approximates attenuation of the radiation, the detector model using a plurality of projection lengths, with each projection length corresponding to a respective material model of a plurality of material models; and
modifying the plurality of projection lengths until the calculated cost function converges to approximate a global minimum of the cost function.

14. The method according to claim 13, wherein the step of optimizing the plurality of projection lengths is further configured to
choose a plurality of random projection lengths, the random projection lengths being chosen to be within a sample space surrounding a plurality of current projection lengths;
assign the plurality of current projection lengths to be equal to the plurality of random projection lengths, when the cost function corresponding to the plurality of random projection lengths is less than the cost function corresponding to the plurality of current projection lengths;
maintain the plurality of current projection lengths unchanged, when the cost function corresponding to the plurality of random projection lengths is greater than or equal to the cost function corresponding to the plurality of current projection lengths; and
repeat the steps of choosing the plurality of random projection lengths, assigning the plurality of current projection lengths, and maintaining the plurality of current projection lengths, until either the cost function corresponding to the plurality of current projection lengths is less than a predetermined threshold or a number of iterations reaches a predetermined maximum number of iterations.

15. The method according to claim 13, wherein the step of calculating a cost function is further configured to calculate the cost function according to the detector model having a linear detector response term and a nonlinear detector response term, wherein the linear and nonlinear detector response terms each include a detector dead time and a radiation flux.

16. The method according to claim 15, wherein the step of calculating a cost function is further configured to calculate the cost function using the radiation flux determined using a reference intensity representing a radiation intensity of the radiation at a radiation source.

17. The method according to claim 13, further comprising:
optimizing the plurality of projection lengths according to a multi-step optimization method having a first step and a second step, wherein
the first step performs a global optimization method solving for the plurality of projection lengths that minimize a first cost function; and
the second step includes using the optimized plurality of projection lengths obtained in the first step to perform a second optimization method solving for the plurality of projection lengths that minimize a second cost function.

18. The method according to claim 13, further comprising reconstructing a plurality of images, wherein each image corresponds a respective material model of a plurality of material models, and the plurality of images are reconstructed using the plurality of projection lengths.

19. The method according to claim 13, wherein the step of optimizing the plurality of projection lengths is further configured to
perform the optimization of the plurality of projection lengths using a stochastic optimization method that is one of a genetic algorithm, a simulated annealing method, a quantum annealing method, a swarm algorithm, an evolutionary algorithm, a random search, a replica exchange method, and a reactive search optimization method.

20. A non-transitory computer readable storage medium including executable instruction, wherein the instructions, when executed by circuitry, cause the circuitry to perform the method according to claim 13.
